(19) United States
(12) Patent Application Publication
(10) Pub. No.: US 2010/0160673 A1
(43) Pub. Date: Jun. 24, 2010

(54) PROCESS FOR THE PREPARATION OF ISOCYANATES IN THE GAS PHASE

(75) Inventors: Rainer Bruns, Leverkusen (DE); Fritz Pohl, Brunsbuttel (DE); Friedhelm Steffens, Leverkusen (DE); Volker Michele, Koln (DE)

Correspondence Address: BAYER MATERIAL SCIENCE LLC, 100 BAYER ROAD, PITTSBURGH, PA, 15205, US

(73) Assignee: Bayer MaterialScience AG, Leverkusen (DE)

(21) Appl. No.: 12/636,850

(22) Filed: Dec. 14, 2009

(30) Foreign Application Priority Data
Dec. 18, 2008 (DE) 10 2008 063 728.9

(51) Int. Cl. C07C 263/10

(52) U.S. Cl. 560/347

(57) ABSTRACT

Primary isocyanates are produced by reacting the corresponding primary amine(s) with phosgene at a temperature above the boiling temperature of the amine(s) in a tube reactor with a reaction space. In this tube reactor, at least one educt stream P containing phosgene and at least one educt stream A containing the amine(s) are fed to the reaction space via a nozzle arrangement. The nozzle arrangement includes a number of n≧1 nozzles aligned parallel to the axis of rotation of the tube reactor and a free space surrounding the nozzles. One of the educt streams A or P is fed to the reaction space via the nozzles and the other educt stream is fed to the reaction space via the free space surrounding the nozzles. The reaction space contains at least one moving mixing device.

### BACKGROUND OF THE INVENTION

The present invention relates to a process for the preparation of primary isocyanates by reaction of the corresponding primary amines with phosgene in the gas phase.

Isocyanates are prepared in large amounts and serve chiefly as starting substances to for the preparation of polyurethanes. They are usually prepared by reaction of the corresponding amines with phosgene. One technique for the preparation of isocyanates is the reaction of the amines with the phosgene in the gas phase. Various processes for the preparation of isocyanates by reaction of amines with phosgene in the gas phase are known from the prior art.

GB-A-1 165 831 describes a process for the preparation of isocyanates in the gas phase in which the reaction of the vaporous amine with phosgene is carried out at temperatures of between 150° C. and 300° C. in a tube reactor which is equipped with a mechanical stirrer and can be temperature-controlled via a heating jacket. The reactor disclosed in GB 1 165 831 is similar to a thin film evaporator, the stirrer of which mixes the gases entering into the reaction space and those present in the reaction space while also brushing the walls of the tube reactor surrounded by the heating jacket in order to prevent a build-up of polymeric material on the tube wall. Such a build-up would make heat transfer difficult. According to the teaching of GB 1 165 831, mixing of the educt streams entering into the reaction space is achieved exclusively by the stirrer running at about 1,000 revolutions per minute. This stirrer is driven externally via a shaft passing through the reactor wall. A disadvantage of the process disclosed in GB-A-1 165 831 is the use of a high-speed stirrer which is driven externally via a shaft which passes through the reactor wall, since when phosgene is employed such a stirrer requires a very high outlay for sealing the reactor for safety reasons. It is another disadvantage that the mixing of the gases is achieved only by the stirrer, which in spite of the high speeds of rotation used leads to long mixing times. These long mixing times result in a wide contact time distribution of the reactants, which according to the teaching of EP-A-570 799 in turn leads to undesirable formation of solids.

EP-A-289 840 describes the preparation of (cyclo)aliphatic diisocyanates by gas phase phosgenation of the amine(s) in a cylindrical space without moving parts in a turbulent flow at temperatures of between 200° C. and 600° C. and over reaction times of the order of 10⁻⁴ seconds. By eliminating moving parts equipped with drives which pass through the reactor wall, the risk of exit of phosgene is reduced. According to the teaching of EP-A-289 840, the gas streams are introduced at one end of the tube reactor through a nozzle and an annular gap between the nozzle and mixing tube in the reactor and are thereby mixed. EP-A-289 840 thus discloses a further development of the mixing technology. The mixing of the gases is advantageously effected by a static mixing device, namely the nozzle and annular gap, instead of the stirrer disclosed in GB 1 165 831. According to the teaching of EP-A-289 840, for it to be possible to carry out the process disclosed therein, it is essential for the dimensions of the tube reactor and the flow rates in the reaction space to be such that a turbulent flow characterized by a Reynolds number of at least 2,500, preferably at least 4,700, prevails in the reaction space. According to the teaching of EP-A-289 840, this turbulence is in general ensured if the gaseous reaction partners pass through the reaction space with a flow rate of more than 90 m/s. Due to the turbulent flow in the cylindrical space (tube), disregarding fluid elements close to the wall, a relatively good flow equipartition in the tube and a relatively narrow dwell time distribution is achieved. According to EP-A-570 799, this leads to a reduction in the formation of solids. A disadvantage of the process disclosed in EP-A-289 840 is that the necessary high flow rates make realization of the dwell time necessary for complete reaction of the amines, especially if aromatic amines are employed, possible only in very long mixing and reactor tubes.

EP-A-570 799 discloses a process for the preparation of aromatic diisocyanates in which the reaction of the associated diamine with the phosgene is carried out in a tube reactor above the boiling temperature of the diamine within an average contact time of the reactants of from 0.5 to 5 seconds. As described in this disclosure, both reaction times which are too long and those which are too short lead to an undesirable formation of solids. A process is therefore disclosed in which the average deviation from the average contact time is less than 6%.

Maintenance of this contact time distribution is achieved by carrying out the reaction in a tubular flow which is characterized either by a Reynolds number of above 4,000 or by a Bodenstein number of above 100. According to the teaching of EP-A-570 799, a plug flow approximating 90% is thereby achieved. All of the volume parts of the flow largely have the same flow times, so that dwell times of all the volume parts are approximately equal and the lowest possible spread of the distribution of the contact time between the reaction partners takes place.

According to the teaching of EP-A-570 799, the deviation from the average contact time when the process is carried out in practice, however, is also essentially determined by the time necessary for mixing the reaction partners. EP-A-570 799 states that as long as the reaction partners are still not mixed homogeneously, gas volumes which have not yet been able to come into contact with the reaction partners are still present in the reaction space and, depending on the mixing, with the same flow times of the volume parts different contact times of the reaction partners are therefore obtained. According to the teaching of EP-A-570 799, mixing of the reaction partners should therefore take place within a period of from 0.1 to 0.3 s to a degree of segregation of 10⁻³, where the degree of segregation serves as a measure of the incompleteness of the mixing (Sec, e.g., Chem.-Ing.-Techn. 44 (1972), p. 1051 et seq.; Appl. Sci. Res.(the Hague) A3 (1953), p. 279). EP-A-570 799 discloses that in principle known methods based on mixing units with moving and static mixing components, preferably static mixing components, can be employed to generate appropriately short, mixing times. According to EP-A-570 799, the use of the jet mixer principle (Chemie-Ing.-Techn. 44 (1972) p. 1055, FIG. 10) in particular delivers sufficiently short mixing times.

In the jet mixer principle (Chemie-Ing.-Techn. 44 (1972) p. 1055, FIG. 10), two educt streams I and II are fed to the tube reactor. Educt stream I is fed via a central nozzle and educt stream II is fed via an annular space between the central nozzle and the tube reactor wall. In this context, the flow rate of the educt stream I is high compared with the flow rate of the educt stream II. After a time which is determined on the basis of the nozzle diameter and on the difference between the flow rates of the educts, or after the corresponding distance, complete mixing of the educts is then achieved.

A disadvantage of the jet mixer principle is that when the reactors, which are often constructed as tube reactors, are increased in size, an increase in the size of the mixing nozzle, which is often constructed as a smooth jet nozzle, also becomes necessary. As the diameter of the smooth jet nozzle increases, however, the speed of mixing of the central jet is reduced due to the larger diffusion path required, and the mixing time is therefore correspondingly lengthened. Furthermore, the risk of back-mixing is increased, which in the case of the reaction of primary amines with phosgene in the gas phase, as stated above, leads to the formation of polymeric impurities and therefore solid caking in the reactor. On conversion of the gas phase phosgenation of primary amines into a process used on a large industrial scale, simple conversion of the geometry to orders of size which are appropriate on a large industrial scale is therefore not possible, because the diameter of the inner tube would have to be increased to such an extent that mixing of the educts in the required short mixing times is no longer possible without additional measures because of the long distances at right angles to the direction of flow Optimization of the use of tube reactors for gas phase phosgenation such as has been disclosed fundamentally in EP-A-570,799 using the jet mixer principle (Chemie-Ing.-Techn. 44 (1972) p. 1055, FIG. 10) is therefore the subject matter of numerous applications aimed at improving the mixing of the educt streams by a further development of the static mixing device.

According to the teaching of EP-A-1 526 129, an increase in the turbulence of the educt stream in the central nozzle has a positive influence on the mixing of the reactants and therefore on the gas phase reaction overall. As a consequence of the better mixing, the tendency towards the formation of by-products decreases and the dwell time and therefore required reactor construction lengths drop significantly. EP-A-1 526 129 discloses a shortening of the mixing zone to 42% of the original length if a spiral coil is employed as a turbulence-generating installed element in the central nozzle.

According to the teaching of EP-A-1 555 258, the disadvantages which arise in the gas phase phosgenation of primary amines from increasing the size of the reactors and the associated increase in the size of the mixing nozzle with the consequence of lengthening of the mixing times can be eliminated if one educt stream is injected in at a high speed via an annular gap located concentrically in the stream of the other educt. According to the teaching of EP-A-1 555 258, this results in small diffusion paths for the mixing and very short mixing times. EP-A-1 555 258 teaches that the reaction of primary amines with phosgene in the gas phase in this way can be carried out with a high selectivity for the desired isocyanate, and a significant reduction in the formation of polymeric impurities and caking. EP-A-1 555 258 also discloses that at comparable speeds of the components at the mixing point, significantly shorter reaction spaces are required to achieve the maximum temperature in the reaction system than when conventional smooth jet nozzles are employed. The reaction of primary amines with phosgene to give the corresponding isocyanates can accordingly be carried out in significantly shorter reactors compared with the prior art. A disadvantage of the process disclosed is that the central stream must be distributed very uniformly over the concentric annular gap and the second educt stream must be distributed very uniformly in the outer and inner annular space to avoid an unstable reaction procedure in the reaction space. This unstable reaction procedure is detectable according to the teaching of EP-A-1 362 847 from variations in temperature and asymmetries in the temperature distribution in the reaction space. The very uniform distribution required for the two educt streams is expensive in construction terms. Further, very small amounts of solids, formation of which cannot be ruled out completely during synthesis of the isocyanates on an industrial scale, lead to blocking of the annular gap and therefore reduce the availability of the isocyanate plant.

According to the teaching of EP-A-1 449 826, the disadvantages resulting from increasing the size of the reactors and the associated increase in the size of the mixing nozzle can be by-passed by dividing the central stream over several nozzles. EP-A-1 449 826 discloses a process for the preparation of diisocyanates by phosgenation of the corresponding diamines, in which the vaporous diamines, optionally diluted with an inert gas or with the vapors of an inert solvent, and phosgene are heated separately to temperatures of from 200° C. to 600° C. and are mixed and reacted in a tube reactor. In this process, a number n≧2 of nozzles aligned parallel to the axis of the tube reactor are arranged in the tube reactor with the stream containing the diamines being fed to the tube reactor via the n nozzles and the phosgene stream being fed to the tube reactor via the remaining free space. According to the teaching of EP-A-1 449 826, a shortening of the mixing times compared with a single nozzle (individual nozzle) with the same cross-sectional area is achieved by the process disclosed therein. Due to the considerably shorter mixing times, there is a positive influence on the distribution of the contact time of the reactants. The considerably shorter mixing times with the same contact time of the reactants make significantly shorter dwell times in the reaction space necessary, and allow the use of reaction spaces of significantly shorter length.

On conversion of the process disclosed in EP-A-1 449 826 into a large-scale industrial dimension, the necessarily short mixing times can be achieved only by correspondingly increased entry speeds of the reactants into the reaction space, such as is required according to the teaching of WO2008/055898 even when alternative nozzle configurations are employed as the mixing device. A disadvantage of increased entry speeds is that the high flow rates for realization of the dwell time necessary for complete reaction of the amines, especially if aromatic amines are employed, is possible only in very long mixing and reactor tubes.

### SUMMARY OF THE INVENTION

The object of the present invention was therefore to make possible a process for the preparation of isocyanates by reaction of primary amines with phosgene in the gas phase on a large industrial scale, which, at the same entry speed of the reactants, includes a faster mixing of the reactants with a simultaneously low risk of blockage of the mixing units.

It has been possible, surprisingly, to achieve this object by a procedure in which the primary amine and the phosgene are reacted in a tube reactor above the boiling temperature of the amine in the gas phase and in which the educt streams enter into the reaction space via a nozzle arrangement whereby one educt stream is fed to the reaction space via n≧1 nozzles aligned parallel to the axis of the reaction space and the second educt stream is fed to the reaction space via the free space surrounding the nozzles. The reaction space contains at least one moving mixing device.

Surprisingly, by combination of the jet mixer principle with a moving mixing device, the increase in the size of the reactors and of the Mixing nozzle/mixing nozzles on a large industrial scale can also be achieved without an increase in the entry speed of the reactants or the use of nozzle configurations which are susceptible to blocking, and with sufficiently short mixing times of the educt streams. The advantageous combination of a static with a moving mixing device was not to be foreseen by the person skilled in the art, since according to the prior art the use of moving mixing devices has not proven to be advantageous for fast gas phase reactions.

### DETAILED DESCRIPTION OF THE INVENTION

The present invention provides a process for the preparation of primary isocyanates by reaction of the corresponding primary amines with phosgene. In this process, the primary amine is reacted with phosgene above the boiling temperature of the amine in a tube reactor. In the tube reactor which comprises the reaction space,
a) at least one educt stream P containing phosgene and at least one educt stream A containing the amine are fed to the reaction space via a nozzle arrangement which includes a number of n≧1 nozzles aligned parallel to the axis of rotation of the tube reactor and a free space surrounding the nozzles, and
b) one of the educt streams A or P is fed to the reaction space via the nozzles and the other educt stream is fed to the reaction space via the free space surrounding the nozzles, and
c) the reaction space contains at least one moving mixing device.

The tube reactor conventionally comprises a reaction space which is essentially rotationally symmetric to the direction of flow. “Rotationally symmetric” in this context means in accordance with the prior art (See, e.g., WO 2007/028 715 A1, p. 3, l. 28 et seq.), that a body or space, here the reaction space, has a rotational symmetry when rotated about the axis of rotation. This can be, for example, a digyric axis of rotation (C2), a trigyric (C3) or a tetragyric axis of rotation (C4), or preferably complete rotational symmetry (C_{∞}). Thus, for example, an area bordered by an ellipse has a digyric axis of rotation. As a further example, an area bordered by a circle has complete rotational symmetry.

Most preferably, the tube reactor employed in the present invention is one having a flow-through cross-sectional area which widens, remains constant and/or decreases, optionally only in sections, in the direction of flow.

Reaction spaces which have flow cross-sections which are oval or composed of any desired closed planar polygons are not preferred, but are in principle also possible.

In the context of this invention, the expression “nozzles aligned parallel to the axis of the tube reactor” is to be understood as meaning that the deviation in angle between the alignment of the central axis of the particular nozzles and the alignment of the central axis of the reactor is less than 5 degrees, preferably less than 3.5 degrees.

In a preferred embodiment of the present invention, the n nozzles aligned parallel to the axis of the tube reactor, when n is a positive integer of greater than 1, preferably have the same diameter. The individual nozzles arc most preferably identical in construction within the framework of production tolerances.

The arrangement of the n nozzles aligned parallel to the axis of the tube reactor, when n is a positive integer of greater than 1, is preferably on a circular ring around the axis of the reactor. If n>2 individual nozzles are employed, in a further embodiment n−1 individual nozzles can be located on a circular ring around a centrally arranged nozzle. In particular, the arrangement of the n nozzles aligned parallel to the axis of the tube reactor is rotationally symmetric, where n is a positive integer of greater than 1.

In another preferred embodiment of the present invention, the n nozzles aligned parallel to the axis of the tube reactor, where n is a positive integer of at least 1, are each connected via a flexible or rigid connecting piece to an inlet for one of the educt streams. Rigid connecting pieces can be pipeline pieces, flexible connecting pieces can be, e.g., hoses or preferably compensators.

In another preferred embodiment of the process of the present invention, the amine (i.e. the at least one educt stream containing the amine) is fed to the reactor via the n nozzles aligned parallel to the axis of the tube reactor, where n is a positive integer of at least 1. In this embodiment, the phosgene (i.e., the at least one educt stream containing the phosgene) is introduced into the space surrounding the nozzles, i.e., into the space demarcated by the reactor wall and the at least one amine nozzle. If the amine stream is fed to the reaction space via only one nozzle, i.e., n=1, this nozzle is preferably positioned centrally on the longitudinal axis of the reaction space in the reactor.

In an alternative embodiment of the process according to the invention, the phosgene (i.e., the at least one educt stream containing the phosgene) is fed to the reactor via the n nozzles aligned parallel to the axis of the tube reactor, where n is a positive integer of at least 1. In this embodiment, the amine (i.e., the at least one educt stream containing the amine) is introduced into the space surrounding the nozzles, i.e. into the space demarcated by the reactor wall and the at least one phosgene nozzle. If the phosgene stream is fed to the reactor via only one nozzle, i.e. n=1, this nozzle is preferably positioned centrally on the longitudinal axis of the reaction space in the reactor.

In the process according to the invention, the educts streams are preferably fed into the reaction space continuously and preferably enter into the reaction space with a speed ratio of from 2 to 20, more preferably from 3 to 15, most preferably from 4 to 12. Preferably, the educt stream which is fed to the reaction space via the n nozzles aligned parallel to the axis of the tube reactor enters into the reactor with the higher flow rate. This educt stream is most preferably the amine-containing educt stream A.

In a particular embodiment of the process according to the invention, the n≧1 nozzles aligned parallel to the axis of the tube reactor or, for n=1, the nozzle preferably positioned centrally in the reactor can be equipped with additional turbulence-generating elements, such as e.g. coils, spiral coils or circular or square plates introduced into the flow at an angle.

In a further preferred embodiment of the process of the present invention, the free space surrounding the nozzles which is demarcated by the reactor wall and the n≧1 nozzles contains at least one, preferably at least two flow equalizers and/or flow rectifiers which equalize the speed of the flow in this space over the entire cross-section of this space. For example, perforated trays, screens, sintered metal, frits or bulk materials, preferably perforated trays, can be employed as flow equalizers. The use of, e.g., honeycomb structures and tube structures as flow rectifiers, as disclosed in EP-A-1 362 847, is likewise possible.

In contrast to a static mixing device, a moving mixing device in the context of the present invention is to be understood as meaning an element which rotates or which moves by oscillation. Examples of suitable mixing devices include stirrers, such as propeller stirrers, angled blade stirrers, disc stirrers, impeller stirrers, cross-arm stirrers, anchor stirrers, blade or grid stirrers, coiled stirrers and toothed disc stirrers. The stirrer can have one or more wings, blades, discs, arms or anchors, which are mounted on a shaft. Wings or blades are preferred. The wings, blades, discs, arms or anchors can be mounted at various positions along the shaft, and they are preferably mounted at the same position along the shaft. They are most preferably mounted at the end of the shaft. Preferably, the moving mixing device has more than one wing or more than one blade. The wings or blades can be set at an angle or straight, and they can have any desired shape or curve.

The speed of rotation of the moving mixing device can be slow or fast, fast being defined as >1,000 revolutions per minute (rpm) and slow being defined as ≦1,000 revolutions per minute. The moving mixing device preferably has a slow speed of rotation.

The at least one moving mixing device can be driven by various methods. In particular, it can be driven by an external drive device or by using the pulse of at least one of the educt streams fed to the reaction space. Most preferably, the at least one moving mixing device is driven in a manner such that the moving fittings of the particular mixing device, for example the shaft, are not led through the reactor wall. This is particularly important from the safety point of view, when hot phosgene gas is employed.

External drive devices in the context of this invention are to be understood as meaning those drive devices which are located outside of the reactor. Examples of suitable external drive devices include motors, in particular electric motors, the drive energy preferably being transmitted to the moving mixing device indirectly (i.e., without a moving element of the moving mixing device being led through the reactor wall). Suitable indirect drive means which may be mentioned here are, for example, magnet-coupled drives.

The pulse of at least one of the educt streams can also be used to drive the at least one moving mixing device. For this, on the one hand the pulse of the educt streams A and/or P which have entered into the reaction space through the n≧1 nozzles aligned parallel to the axis of rotation of the tube reactor and/or through the free space surrounding the nozzles can be used to drive the moving mixing device, i.e. in this case the pulse of the flow in the reaction space is used to drive the moving mixing device in the reaction space. The at least one moving mixing device in the reaction space can also preferably be connected via at least one shaft to a drive propeller, the drive propeller being outside the reaction space. Preferably, the drive propeller is in the direction of flow before entry into the reaction space, and in particular in the educt stream A and/or the educt stream P. In a particularly preferred embodiment, the drive propeller is in the educt stream fed in via the n≧1 nozzles aligned parallel to the axis of rotation of the tube reactor. In another preferred embodiment, the drive propeller is in the educt stream which is fed in through the region of the free space surrounding the nozzles.

The drive propeller can have one or more wings, blades, discs, arms or anchors. Wings or blades are preferred. Preferably, the drive propeller has more than one wing or more than one blade, and these are preferably mounted on the shaft at an angle.

If more than one moving mixing device is employed, the several moving mixing devices are preferably driven by the same method, i.e. all the moving mixing devices are preferably driven by an external drive device or using the pulse of at least one of the educt streams. Preferably, each moving mixing device has a separate drive propeller, but it is also conceivable that one drive propeller drives several moving mixing devices. Preferably, an external drive device drives only one moving mixing device, but it is also conceivable that one external drive device could drive several moving mixing devices. Preferably, each moving mixing device is connected to one drive propeller, but it is also conceivable that each moving mixing device is driven by more than one drive propeller.

The moving mixing device is in the reaction space. In the context of the present invention, the reaction space starts with the exit of the flow in the direction of flow from the n≧1 nozzles aligned parallel to the axis of the tube reactor, where n is a positive integer of at least 1. With the exit of the flow from the n≧1 nozzles aligned parallel to the axis of the tube reactor mixing of the educt streams starts, due to the speed of the reactions in the gas phase, phosgenation of primary amine begins immediately.

The at least one moving mixing device may be located at any desired position in the reaction zone. Preferably, the at least one moving mixing device is less than 5×D in the direction of flow away from the start of the reaction space, most preferably less than 3×D. In this context, D repressents the largest diameter of the reaction space at the level of the exit opening from the nozzle. If several moving mixing devices are present in the reaction zone, they preferably have the same position in the reaction zone.

The moving mixing device can be centrally located on the axis of the reactor, but an eccentric location of the moving mixing device with respect to the axis of the reactor is also conceivable.

If more than one moving mixing device is employed in the reaction space, they are preferably located on a circular ring around the axis of the reactor. In a further embodiment, the moving mixing devices can be located on a circular ring around a centrally arranged moving mixing device. If more than one moving mixing device is employed, the arrangement thereof is preferably symmetric.

In another preferred embodiment, the reactor has n>1 nozzles aligned parallel to the axis of the reaction space and m≧1 moving mixing devices, where n and m are each positive integers, the entire arrangement preferably being symmetric. In a further particularly preferred embodiment, the arrangement of the n>1 nozzles aligned parallel to the axis of the reaction space and m≧1 moving mixing devices, where n and m are each positive integers, is preferably symmetric with respect to the axis of the reactor.

The wings, blades, discs, arms or anchors of the at least one moving mixing device can have various lengths. If a reaction space characterized by a complete rotational symmetry is used, the maximum length is limited by half the diameter of the reactor. In contrast, if a reaction space characterized by a C2 symmetry is employed, the maximum length of these fittings results from half the diameter of the shorter reactor diameter. Preferably, the wings, blades, discs, arms or anchors are a distance of 0.01×D, most preferably 0.1×D, where D has the meaning defined above, from the wall of the reaction space.

By the at least one moving mixing device in the reaction space, the mixing of the educts, of which the one educt stream is fed to the reaction space via the n≧1 nozzles aligned parallel to the axis of the reaction space and the second educt stream is fed to the reaction space via the free space which remains, is improved.

In the case of arrangement of the at least one moving mixing device on the axis of the at least one nozzle located parallel to the axis of the reaction space, the improved mixing is due to the fact that the educt jet leaving the nozzle aligned parallel to the axis of the tube reactor diverges and therefore mixes more quickly with the educt stream leaving the free space.

In the case of the embodiment where the reactor has n>1 nozzles aligned parallel to the axis of the reaction space and m≧1 moving mixing devices (where n and m are each positive integers and are preferably arranged symmetrically with respect to the axis of the reactor), the m≧1 moving mixing devices have the effect of intensifying the mixing of the educt gas streams by increasing the turbulence and twisting the flow.

Because of the at least one moving mixing device in the reaction space, it is possible to increase the diameter of the nozzles with the same entry speed of the reactants without a reduction in the mixing speed of the jet thereby taking place, and without the negative consequences of lengthening the mixing time and extending the contact time. It is particularly surprising that a slow-running moving mixing device generates adequate additional turbulence that the mixing zone may be shortened by up to 40%.

Primary amines which can preferably be converted into the gas phase without decomposition can be used in the process according to the invention. Amines, in particular diamines, based on aliphatic or cycloaliphatic hydrocarbons having 1 to 15 carbon atoms are particularly suitable. Especially suitable amines are 1,6-diamino-hexane, 1-amino-3,3,5-trimethyl-5-aminomethylcyclohexane (IPDA) and 4,4′-diaminodicyclohexylamine. 1,6-Diaminohexane (HAD) is most preferably used.

Aromatic amines which can preferably be converted into the gas phase without decomposition may likewise preferably be used in the process of the present invention. Examples of preferred aromatic amines are toluenediamine (TDA), in particular 2,4-TDA and 2,6-TDA and mixtures thereof; diaminobenzene; naphthyldiamine (NDA); and 2,2′-, 2,4′- or 4,4′-methylenediphenyldiamine (MDA) or isomer mixtures thereof. Toluenediamine (TDA), in particular 2,4-TDA and 2,6-TDA and mixtures thereof, is most preferred.

Before carrying out the process of the present invention, the starting amine as a rule is vaporized and heated to from 200° C. to 600° C., preferably from 200° C. to 500° C., most preferably from 250° C. to 450° C., and is optionally fed to the reaction space in a form diluted with an inert gas, such as N₂, He or Ar, or with the vapor of an inert solvent, e.g., an aromatic hydrocarbon, optionally with halogen substitution, such as chlorobenzene or o-dichlorobenzene.

The vaporization of the starting amine can be carried out in any of the known vaporization apparatuses. Preferred vaporization systems are those in which a small work content is led with a high circulating output over a falling film evaporator and, to minimize exposure of the amine to heat, inert gas or vapors of an inert solvent are optionally fed into the system.

In one of the most preferred embodiments of the present invention, vaporization systems in which a small work content is circulated over at least one micro-heat exchanger or micro-evaporator are employed. The use of appropriate heat exchangers for vaporization of amines is disclosed, e.g., in EP-A-1 754 698. The apparatuses disclosed in paragraphs [0007] to [0008] and [0017] to [0039] of EP-A-1 754 689 are preferably employed in the process of the present invention.

The vaporous amine(s) can still contain non-vaporized droplets of the amine(s) (aerosols). However, the vaporous amine preferably contains essentially no droplets of non-vaporized amine, i.e., not more than 0.5 wt. % of the amine, most preferably not more than 0.05 wt. % of the amine, based on the total weight of amine, is present in the form of non-vaporized droplets and the remaining part of the amine is present in vaporous form. Most preferably, the vaporous amine contains no droplets of non-vaporized amine.

The vaporization and superheating of the starting amine is preferably carried out in several stages in order to avoid non-vaporized droplets in the vaporous amine stream. Multi-stage vaporization and superheating steps in which droplet separators arc incorporated between the vaporization and superheating systems and/or the vaporization apparatuses which also function as a droplet separator are particularly preferred. Suitable droplet separators are described, e.g., in “Droplet Separation”, A. Bürkholz; VCH Verlagsgesellschaft, Weinheim—New York—Basel—Cambridge, 1989. After leaving the last superheater in the direction of flow, the vaporous amine which has been preheated to its intended temperature is fed with an average dwell time of preferably from 0.01 to 60 s, more preferably from 0.01 to 30 s, most preferably 0.01-15 s, to the reactor or the nozzle arrangement for reaction. The risk of a renewed formation of droplets is counteracted in this case via technical measures, e.g., an adequate insulation to avoid losses by radiation. The reactor running time is increased significantly by generation of an essentially droplet-free vaporous amine stream before entry into the reactor.

In the process of the present invention, it is advantageous to employ phosgene in excess with respect to the amine groups to be reacted. Preferably, a molar ratio of phosgene to amine groups of from 1.1:1 to 20:1, preferably from 1.2:1 to 5:1 is present. The phosgene is also heated to a temperature of from 200° C. to 600° C. and optionally fed to the reaction space in a form diluted with an inert gas, such as N₂, He or Ar, or with the vapors of an inert solvent (e.g., an aromatic hydrocarbon, without or with halogen substitution, such as chlorobenzene or o-dichlorobenzene).

In the process of the present invention, the separately heated reactants arc introduced as described above into the reaction space of a tube reactor via a nozzle arrangement and are preferably reacted adiabatically taking into account suitable reaction times. The isocyanate is then preferably condensed by cooling the reaction mixture to a temperature above the decomposition temperature of the corresponding carbamic acid chloride.

The necessary dwell time for complete reaction of the amine with the phosgene to give the corresponding isocyanate is between 0.05 and 15 seconds, depending on the nature of the amine employed, the start temperature, the adiabatic increase in temperature in the reaction space, the molar ratio of amine to phosgene, any dilution of the reaction partners with inert gases and the reaction pressure chosen.

If the minimum dwell time for the complete reaction for the particular system (determined on the basis of the amine employed, start temperature, adiabatic increase in temperature, molar ratio of the reactants, dilution gas, reaction pressure) is exceeded by less than 20%, preferably less than 10%, the formation of secondary reaction products, such as isocyanurates and carbodiimides can be largely avoided.

Preferably, neither the reaction space nor the nozzle arrangement has heating surfaces, which can give rise to exposure to heat and cause secondary reactions, such as isocyanurate or carbodiimide formation, or cooling surfaces, which can give rise to condensation causing formation of deposits. The phosgene and amine educts arc preferably reacted adiabatically in this way, apart from any losses by radiation and conduction. In this context, the adiabatic increase in temperature in the mixing unit and reactor is established solely via the temperatures, compositions and relative meterings of the educt streams and the dwell time in the mixing units and the reactors.

In a preferred embodiment of the process of the present invention, the throughput capacity of the reactor employed under the required reaction conditions is >1 t of amine/h, preferably 2-50 t of amine/h, most preferably 2-12 t of amine/h. These values most preferably apply to toluenediamine. In this context, throughput capacity means that the stated throughput capacity of amine per h can be reacted in the reactor.

After the phosgenation reaction has taken place in the reaction space, the gaseous reaction mixture, which preferably includes at least an isocyanate, phosgene and hydrogen chloride, is preferably freed from the isocyanate formed. This can be carried out, for example, by subjecting the mixture leaving the reaction space continuously to a condensation in an inert solvent after leaving the reaction space, as has already been recommended for other gas phase phosgenation processes (EP-A-0 749 958).

Preferably, however, the condensation is carried out by a procedure in which the reaction space employed in the process of the present invention has at least one zone into which one or more suitable streams of liquid (“quench liquids”) are sprayed for discontinuation of the reaction of the amines and the phosgene to give the corresponding isocyanates. By this means, as described in EP-A-1 403 248, rapid cooling of the gas mixtures can be carried out without the use of cold surfaces.

In a particularly preferred embodiment of the process of the present invention, at least one zone (cooling zone) is integrated into a quenching stage, such as has been disclosed e.g. in EP-A-1 403 248. In an especially preferred embodiment of the present invention, two or more cooling zones are employed, and these cooling zones are integrated and operated with a quenching stage, as disclosed with respect to construction and operation in EP-A-1 935 875.

Instead of the integrated combination of one or more cooling zones of a reactor with one or more quenching stages (disclosed in EP-A-1 935 875), an integrated combination of the cooling zones of several reactors with a quenching stage is also possible. However, the integrated combination of a reactor with at least one cooling zone with a quenching stage is preferred.

Regardless of the nature of the cooling process chosen, the temperature of the at least one cooling zone is preferably chosen so that it is above the decomposition temperature of the carbamoyl chloride corresponding to the isocyanate. The isocyanate and, where appropriate, the solvent co-used as a diluent in the amine vapor stream and/or phosgene stream should condense to the greatest extent or dissolve in the solvent to the greatest extent, while excess phosgene, hydrogen chloride and inert gas optionally co-used as a diluent pass through the condensation or quenching stage to the greatest extent without being condensed or dissolved. Solvents kept at a temperature of from 80 to 200° C., preferably from 80 to 180° C. (for example, chlorobenzene and/or dichlorobenzene), or isocyanate or mixtures of the isocyanate with chlorobenzene and/or dichlorobenzene kept in these temperature ranges are particularly suitable for selective isolation of the isocyanate from the gaseous reaction mixture. On the basis of the physical data at a given temperature, pressure and composition, the person skilled in the art can easily estimate what weight content of the isocyanate condenses in the quenching or passes through this without being condensed. It is likewise easy to estimate what weight content of the excess phosgene, hydrogen chloride and inert gas optionally used as a diluent passes through the quenching without being condensed or dissolves in the quenching liquid.

Generation of the flow of the gaseous reaction mixture as a flow through the reaction space without substantial back-mixing, which is preferred for the process of the present invention, is ensured by a pressure gradient over the reaction space. The pressure gradient preferably exists between the educt feed lines before the mixing and the exit from the condensation or quenching stage. Preferably, the absolute pressure in the educt feed lines before the mixing is 200 to 3,000 mbar and after the condensation or quenching stage is 150 to 2,500 mbar. However, it is advisable to maintain a pressure difference from the educt feed lines via the reaction space to after the condensation or quenching stage of preferably at least 50 mbar for the purpose of ensuring the directed flow mentioned and a good mixing of the educts.

The gas mixture leaving the condensation or quenching stage is preferably freed from residual isocyanate in a downstream gas wash with a suitable wash liquid, and is preferably then freed from excess phosgene in any manner known to be suitable by those skilled in the art. This can be carried out by means of a cold trap, absorption in an inert solvent (e.g., chlorobenzene or dichlorobenzene) or by adsorption and hydrolysis on active charcoal. The hydrogen chloride gas passing through the phosgene recovery stage can be recycled in any manner known to be suitable for recovery of the chlorine required for the synthesis of phosgene. The wash liquid obtained after its use for the gas wash is then preferably at least partly employed as the quench liquid for cooling the gas mixture in the corresponding zone of the reaction space.

The isocyanates are subsequently preferably prepared in a pure form by working up the solutions or mixtures from the condensation or quenching stage by distillation.

The following Examples are given to illustrate specific embodiments of the present invention.

### EXAMPLES

### Example 1

### Cold Flow Model without a Stirrer

Air is flowed through a tube of 54 mm internal diameters under ambient conditions at a speed of 5.5 m/s. The tube ended in a nozzle with a diameter of 40 mm, and in the nozzle the air speed was 10 m/s. The air exited the nozzle as a free jet into an open half-space. To determine the effect jet divergence angle, a mist aerosol was added to the air flow via an injector, and a jet diameter of 167 mm was measured by means of a video measuring technique at a position 717 mm downstream of the nozzle mouth. When converted, this corresponded to an effective divergence angle of the nozzle jet of 10.1° (total angle). The effective divergence angle determined in this way was used as a measure of the mixing efficiency of the nozzle. Assuming a given external flow (diameter of the annular space around the nozzle), it allowed calculation of the mixing path from the jet and external flow.

### Example 2

### Cold Flow Model with a Stirrer

Air flowed through a tube of 54 nun internal diameter such as that employed in Example 1 under ambient conditions with a speed of 5.5 m/s. On the axis of the tube was a rotatably mounted shaft, on the end of which facing the flow was fixed a propeller with six blades set at an angle of 45° and a diameter of 50 mm. Downstream of the propeller the tube ended in a nozzle with a diameter of 40 mm, and the air exited the nozzle as a free jet into an open half-space. The shaft on the axis of the tube extended to a position 20 mm downstream of the nozzle mouth. At this point, a stirrer with 6 blades aligned parallel to the direction of flow of the gas exiting the nozzle mouth and a diameter of 40 mm was mounted. The propeller on the end of the shaft facing the flow was caused to move by the flow and transmit this movement via the shaft to the stirrer downstream of the nozzle. By the centrifugally conveying stirrer, the air exiting the nozzle with an axial speed of 10 m/s acquired a radial speed component directed outwards from the axis of rotation. To determine the effective jet divergence angle, a mist aerosol was added to the air flow upstream of the drive propeller via an injector, and a jet diameter of 253 mm was measured by means of a video measuring technique at a position 717 mm downstream of the nozzle mouth. When converted, this corresponded to an effective divergence angle of the nozzle jet of 16.9° (total angle). Assuming a given external flow (diameter of the annular space around the nozzle), this significantly greater divergence angle of the nozzle jet compared with that of Example 1 led to a correspondingly shorter mixing path of the jet with this external flow. At a diameter of the annular space around the nozzle which was assumed to be constant, the ratio of the mixing path lengths were be calculated according to the following formula:

$$\frac{mixing path length Example 2}{mixing path length Example 1} = \frac{tan ( 0.5 \cdot divergence angle Example 1 )}{tan ( 0.5 \cdot divergence angle Example 2 )}$$

In this case, the mixing path length for Example 2 was only 60% of the mixing path length for Example 1, that is to say the stirrer had the effect of shortening the mixing path length by 40%.

### Example 3

### Phosgenation of TDA (According to the Invention)

1.9 t/h of a mixture of vaporous 2,4- and 2,6-toluenediamine (80:20) as educt stream A was fed via a nozzle, and phosgene with gaseous HCl as educt stream P was fed via the free space surrounding the nozzle to a rotationally symmetric reaction space. The educt streams A and P were each heated separately to above 300° C. Downstream of the nozzle the reaction space had a stirrer which was fixed to the nozzle via a mounted shaft, the stirrer being driven by the pulse of the flow exiting the nozzle. The shaft did not pass through the reactor wall. The stirrer included 6 blades distributed uniformly over a circle. The reaction in the reaction space took place adiabatically within a dwell time of less than 10 seconds, a reactor exit temperature of approx. 430° C. was established. The gas mixture was passed through a condensation stage and was thereby cooled to a gas temperature of approx. 165° C. The condensate obtained was fed to a distillation sequence and gave pure TDI. The non-condensed gas mixture was washed with o-dichlorobenzene in a subsequent washing and the by-product HCl was separated from the excess phosgene by absorption. The o-dichlorobenzene obtained in the washing was employed in the condensation step.

The pressure difference between the pressure in the TDA feed line and the pressure at the gas exit from the condensation stage was 10 mbar, in order to achieve a directed gas flow from the feed lines.

After an experimental time of 200 hours, the pressure difference was 11 mbar and was therefore unchanged within the context of measurement accuracy of large-scale industrial measuring instruments. An inspection showed no deposits of solids.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of a primary isocyanate comprising reacting a primary amine with phosgene in a tube reactor at a temperature above the boiling point of the primary amine wherein the tube reactor comprises a reaction space in which
a) at least one educt stream P containing the phosgene and at least one educt stream A containing the amine are fed to the reaction space via a nozzle arrangement, wherein the nozzle arrangement includes one or more nozzles aligned parallel to the axis of rotation of the tube reactor and a free space surrounding the nozzles, and
b) one of the educt streams A or P is fed to the reaction space via the nozzle or nozzles and the other educt stream is fed to the reaction space via the free space surrounding the nozzle or nozzles, and
c) the reaction space contains at least one moving mixing device.

2. The process of claim 1 in which the at least one moving mixing device is a stirrer.

3. The process of claim 1 in which the at least one mixing device has no moving fittings passing through the tube reactor wall.

4. The process of claim 1 in which the at least one mixing device has no external drive.

5. The process of claim 1 in which the at least one mixing device is driven by pulsation of at least one of the educt streams A or P.

6. The process of claim 1 in which the at least one mixing device is connected to an external drive via a magnet coupling.

7. The process of claim 1 in which the at least one educt stream A containing the amine is fed to the reaction space via the nozzle or nozzles and the at least one educt stream P containing the phosgene is fed to the reaction space via the free space surrounding the nozzle or nozzles.

8. The process of claim 1 in which the reaction of the amine with the phosgene is carried out by an adiabatic reaction procedure.

9. The process of claim 1 in which the reaction space is essentially rotationally symmetric and has over its entire length a flowed-through cross-sectional area which widens, remains constant and/or decreases in the direction of flow.

10. The process of claim 1 in which the reaction space is essentially rotationally symmetric and has sections in which a flowed-through cross-sectional area widens, remains constant and/or decreases in the direction of flow.

11. The process of claim 1 in which diaminohexane, isophoronediamine, 2,4- and/or 2,6-toluenediamine, methylenediphenyldiamine, naphthyldiamine or a mixture thereof is employed as the primary amine.

12. The process of claim 1 in which the reactor has a throughput capacity of >1 t of amine/h.
